(19) United States
(12) Patent Application Publication
(10) Pub. No.: US 2025/0295925 A1
(43) Pub. Date: Sep. 25, 2025

(54) FIRMWARE INDEPENDENT RESET

(71) Applicant: Cochlear Limited, Macquarie University, NSW (AU)

(72) Inventor: Helmut Christian EDER, Bemboka, NSW (AU)

(21) Appl. No.: 18/869,171

(22) PCT Filed: Jun. 14, 2023
(86) PCT No.: PCT/IB2023/056107

§ 371 Date: Nov. 25, 2024

Related U.S. Application Data
(60) Provisional application No. 63/367,242, filed on Jun. 29, 2022.

(51) Int. Cl. A61N 1/372; A61N 1/36; A61N 1/378; G16H 40/63

(52) U.S. Cl. CPC A61N 1/37223; A61N 1/36038; A61N 1/3787; G16H 40/63

(57) ABSTRACT

An apparatus includes at least one housing configured to be implanted within a recipient's body. The apparatus further includes communication circuitry, control circuitry, stimulation circuitry, and reset circuitry within the at least one housing. The communication circuitry is configured to wirelessly communicate, via a transcutaneous wireless communication link, with a device external to the recipient's body. The control circuitry is configured to generate control signals in response to power and/or data signals received via the transcutaneous wireless communication link. The stimulation circuitry is configured to respond to the control signals by providing stimulation and/or at least one medicament to the recipient's body. The reset circuitry is configured to respond to reset signals received via the transcutaneous wireless communication link by resetting the control circuitry and/or the stimulation circuitry to a default operational state.

### BACKGROUND

### Field

The present application relates generally to systems and methods for controlling a device implanted on or within a recipient's body.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises at least one housing configured to be implanted within a recipient's body. The apparatus further comprises communication circuitry, control circuitry, stimulation circuitry, and reset circuitry within the at least one housing. The communication circuitry is configured to wirelessly communicate, via a transcutaneous wireless communication link, with a device external to the recipient's body. The control circuitry is configured to generate control signals in response to power and/or data signals received via the transcutaneous wireless communication link. The stimulation circuitry is configured to respond to the control signals by providing stimulation and/or at least one medicament to the recipient's body. The reset circuitry is configured to respond to reset signals received via the transcutaneous wireless communication link by resetting the control circuitry and/or the stimulation circuitry to a default operational state.

In another aspect disclosed herein, a method comprises wirelessly receiving, using an implant within a recipient's body, signals transmitted through tissue from a device external to a recipient's body, the implant comprising control circuitry and an internal power source. The method further comprises, in response to the signals, controlling the implant while the implant is in at least one functional state in which the internal power source is operationally engaged with the control circuitry. The method further comprises detecting, using the implant, a predetermined modulation of the signals while the implant is in a malfunctioning state. The method further comprises responding to the detected predetermined modulation of the signals by transitioning the implant from the malfunctioning state to a reset state in which the internal power source is operationally disengaged from the control circuitry.

In another aspect disclosed herein, a system comprises a first portion configured to be worn externally on a recipient's body. The device is configured to generate electromagnetic carrier waves with time-varying modulations. The system further comprises a second portion configured to be implanted within the recipient's body. The second portion is configured to transcutaneously receive at least a portion of the electromagnetic carrier waves from the first portion. The second portion comprises at least one microprocessor configured to execute firmware configured to control operation of the second portion in response to the electromagnetic carrier waves received from the first portion. The second portion further comprises reset circuitry configured to operate independently from operation of the firmware. The reset circuitry is further configured to monitor the time-varying modulations for a pattern indicative of a reset signal from the first portion and to respond to detection of the pattern on the electromagnetic carrier waves by resetting the at least one microprocessor.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is a perspective view of an example fully implantable middle ear implant auditory prosthesis implanted in a recipient in accordance with certain implementations described herein;

FIG. 3 schematically illustrates an example external device and an example implanted apparatus in accordance with certain implementations described herein;

FIG. 4 schematically illustrates an example predetermined temporal profile indicative of a reset signal in accordance with certain implementations described herein; and

FIG. 5 is a flow diagram of an example method in accordance with certain implementations described herein.

### DETAILED DESCRIPTION

Certain implementations described herein provide a firmware independent reset of an implant (e.g., a reset that can be invoked by communication from an external device via the transcutaneous wireless power and/or data communication link) regardless of the state of the microprocessor executing firmware of the implant. Such a firmware independent reset can be used to reset the implant (e.g., if the implant begins behaving in an unintended manner) without reliance on correct firmware operation (e.g., since the firmware may not be operating correctly in such an erroneous state of operation). By having the reset signal received by the implant decoded by dedicated hardware that cannot be affected by any firmware programmable parameters, the firmware is unable to disable or otherwise affect the reset operation. For implants that are powered by an internal power source (e.g., battery), the firmware independent reset is configured to disengage (e.g., disconnect) the internal power source from other circuitry of the implant, without the need for firmware intervention and with the firmware unable to prevent and/or interfere with the reset operation.

The teachings detailed herein are applicable, in at least some implementations, to any type of implantable or non-implantable stimulation system or device (e.g., implantable or non-implantable auditory prosthesis device or system). While certain implementations are described herein in the context of auditory prosthesis devices, certain other implementations are compatible in the context of other types of medical devices that can utilize the teachings detailed herein and/or variations thereof (e.g., neurostimulators; pacemakers; other medical implants comprising an implanted power source).

Merely for ease of description, apparatus and methods disclosed herein are primarily described with reference to an illustrative medical device, namely an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

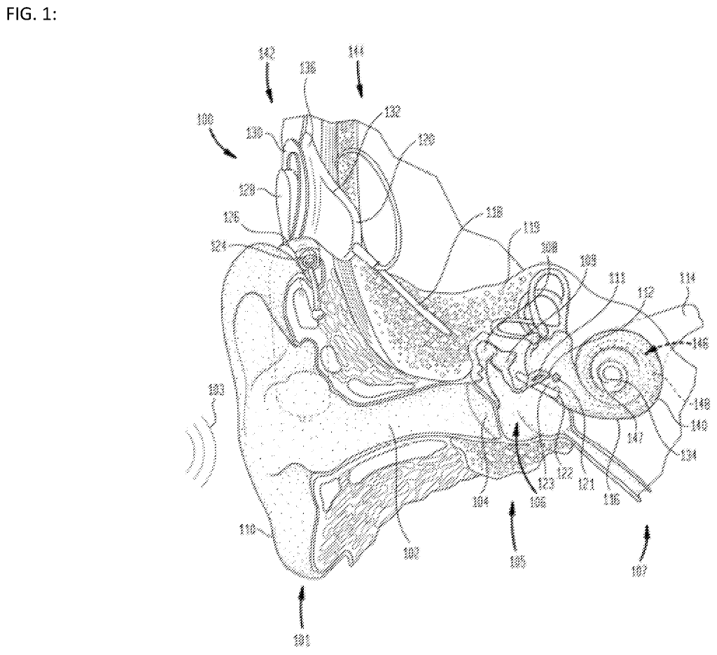

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable microphone assembly, as described more fully herein. In certain implementations, the example cochlear implant auditory prosthesis 100 of FIG. 1 can be in conjunction with a reservoir of liquid medicament as described herein.

As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery or other power storage device (e.g., circuitry located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

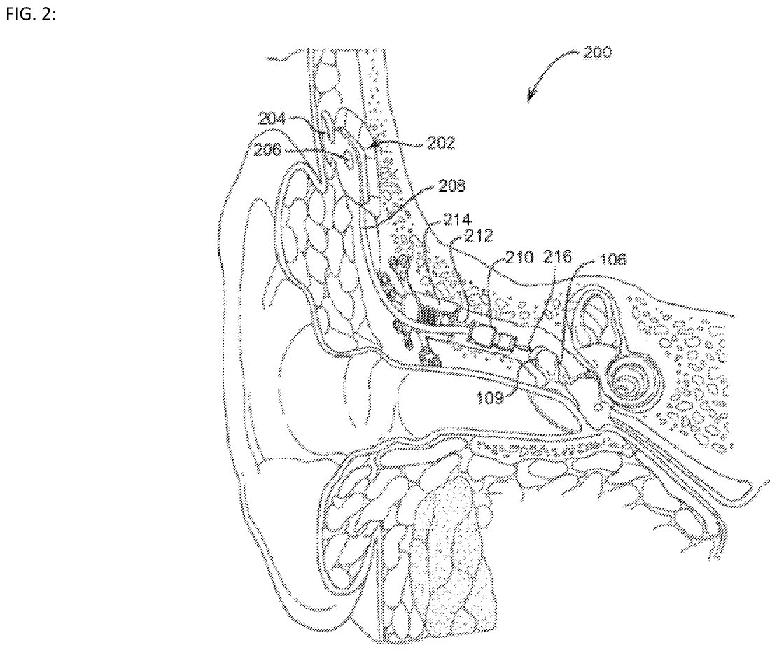

FIG. 2 schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 2 comprises a biocompatible implantable assembly 202 (e.g., comprising an implantable capsule) located subcutaneously (e.g., beneath the recipient's skin and on a recipient's skull). While FIG. 2 schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include a power storage device (e.g., battery or other power storage circuitry) and a signal processor (e.g., a sound processing unit). Various additional processing logic and/or circuitry components can also be included in the implantable assembly 202 as a matter of design choice.

For the example auditory prosthesis 200 shown in FIG. 2, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1 and 2 can comprise an implantable microphone assembly, such as the microphone assembly 206 shown in FIG. 2. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 2 is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1 utilizes an external microphone 124 and the auditory prosthesis 200 shown in FIG. 2 utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1 and 2 are merely illustrative.

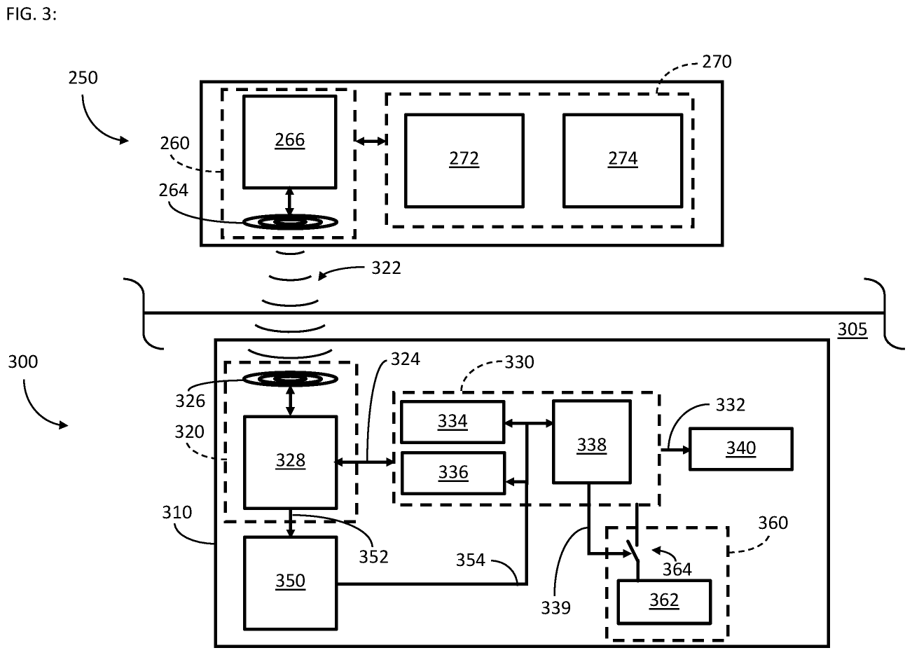

FIG. 3 schematically illustrates an example external device 250 and an example implanted apparatus 300 in accordance with certain implementations described herein. In certain implementations, the external device 250 and the implanted apparatus 300 are components of a stimulation system configured to provide stimulation signals to the recipient. For example, for a sensory stimulation system (e.g., auditory prosthesis system; visual prosthesis system), the stimulation signals can be configured to be received and perceived by the recipient as sensory information. For another example, for a neurostimulation system, the stimulation signals can be configured to be applied to selected portions of the recipient's nervous system (e.g., brain; spinal cord) to treat various maladies (e.g., epilepsy; Alzheimer's disease; Parkinson's disease; chronic pain). For still another example, for a muscle stimulation system, the stimulation signals can be configured to be applied to selected portions of the recipient's musculature system (e.g., legs; arms; torso; heart; tongue) to treat various maladies. In certain other implementations, the external device 250 and the implanted apparatus 300 are components of an implantable micropump system configured to controllably administer at least one medicament to a portion of the recipient's body 305 or to controllably draw fluid from a portion of the recipient's body 305.

In certain implementations, as schematically illustrated by FIG. 3, the device 250 is configured to be worn externally by a recipient (e.g., outside and/or on the recipient's body 305) and comprises external communication circuitry 260 (e.g., comprising at least one antenna 264 and wireless communications interface circuitry 266) and external functional circuitry 270 (e.g., comprising at least one microcontroller 272) configured to control operation of the device 250 (e.g., in response to user input). In certain implementations, the at least one antenna 264 comprises multiple turns of electrically insulated single-strand or multi-strand metal wire (e.g., a planar electrically conductive wire with multiple windings having a substantially circular, rectangular, spiral, or oval shape or other shape) or metal traces on epoxy of a printed circuit board. The external communication circuitry 260 is configured to generate and transmit time-modulated electromagnetic carrier waves to the apparatus 300, through at least a portion of the recipient's body 305 to form a transcutaneous wireless communication link 322 to the apparatus 300. For example, the device 250 can comprise an external component 142 of an auditory prosthesis 100, 200, the external communication circuitry 260 can comprise an external transmitter unit 128, and the external functional circuitry 270 can comprise an external microphone 124 and a sound processing unit 126. In addition, the device 250 can comprise a power source (not shown).

In certain implementations, as schematically illustrated by FIG. 3, the apparatus 300 comprises at least one housing 310 configured to be implanted within a recipient's body 305. The apparatus 300 further comprises, within the at least one housing 310, communication circuitry 320, control circuitry 330, stimulation circuitry 340, and reset circuitry 350. The communication circuitry 320 is configured to wirelessly communicate, via the transcutaneous wireless communication link 322, with the device 250 external to the recipient's body 305 (e.g., external component 142 of the auditory prosthesis 100; external component of the auditory prosthesis 200). The control circuitry 330 (e.g., at least one microcontroller) is configured to generate control signals 332 in response to power and/or data signals 324 received via the transcutaneous wireless communication link 322. The stimulation circuitry 340 is configured to respond to the control signals 332 by providing stimulation and/or at least one medicament to the recipient's body 305. The reset circuitry 350 is configured to respond to reset signals 352 received via the transcutaneous wireless communication link 322 by resetting the control circuitry 330 and/or the stimulation circuitry 340 to a default operational state.

In certain implementations, the at least one housing 310 of the implantable apparatus 300 is configured to be positioned beneath tissue of the recipient's body 305. For example, the at least one housing 310 can be beneath the skin, fat, and/or muscular layers and above a bone (e.g., skull) in a portion of the recipient's body 305 (e.g., the head). In certain implementations, the at least one housing 310 is configured to hermetically seal the communication circuitry 320, control circuitry 330, stimulation circuitry 340, and reset circuitry 350 from an environment surrounding the at least one housing 310. The at least one housing 310 of certain implementations comprises at least one biocompatible material (e.g., polymer; silicone) that is substantially transparent to the electromagnetic carrier waves generated by the external device 250 such that the at least one housing 310 does not substantially interfere with the transmission of the electromagnetic carrier waves via the transcutaneous wireless communication link 322.

The at least one housing 310 can have a length and/or width (e.g., along one or two lateral directions substantially parallel to the recipient's skin and/or bone surface) that is less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters). The at least one housing 310 can have a thickness (e.g., along a direction substantially perpendicular to the recipient's skin and/or bone surface) less than or equal to 10 millimeters (e.g., in a range of less than or equal to 7 millimeters, in a range of less than or equal to 6 millimeters; in a range of less than or equal to 5 millimeters).

In certain implementations, the apparatus 300 comprises at least one internal magnetic (e.g., ferromagnetic; ferrimagnetic; permanent magnet) element (e.g., disk; plate) positioned within the at least one housing 310 and the external device 250 comprises at least one external magnetic (e.g., ferromagnetic; ferrimagnetic; permanent magnet) element (e.g., disk; plate) positioned within an external housing. The at least one internal magnetic element and the at least one external magnetic element can be configured to establish a magnetic attraction between the external device 250 and the apparatus 300 sufficient to hold the external device 250 against an outer surface of the recipient's body 305 (e.g., skin).

In certain implementations, the communication circuitry 320 comprises at least one antenna 326 and analog interface circuitry 328 in electrical communication with the at least one antenna 326. The at least one antenna 326 is configured to be in wireless communication with the at least one antenna 264 of the external communication circuitry 260 of the external device 250. In certain implementations, the at least one antenna 326 comprises multiple turns of electrically insulated single-strand or multi-strand metal wire (e.g., a planar electrically conductive wire with multiple windings having a substantially circular, rectangular, spiral, or oval shape or other shape) or metal traces on epoxy of a printed circuit board. For example, the at least one antenna 326 can comprise at least one internal radio-frequency (RF) antenna in operative communication with at least one external RF antenna of the device 250 to form the transcutaneous wireless communication link 322, which can have multiple frequency channels and can be configured to transfer power and/or data signals from the external device 250 to the apparatus 300. For another example, the at least one antenna 326 can comprise at least one internal magnetic induction (MI) antenna in operative communication with at least one external MI antenna of the device 250 to form the transcutaneous wireless communication link 322, which can be configured to transfer data signals but not power signals from the external device 250 to the apparatus 300 (e.g., over a distance that does not exceed 20 cm). The signals transmitted via the transcutaneous wireless communication link 322 can have one or more carrier frequencies in a range of 2 MHz to 6 GHz (e.g., in a range of 2 MHz to 10 MHz; in a range of 10 MHz to 30 MHz; in a range of 30 MHz to 1 GHz; in a range of 1 GHz to 6 GHz; about 5 MHz; about 22.7 MHz; about 2.4 GHz).

In certain implementations, the control circuitry 330 comprises at least one microcontroller 334 and other digital control circuitry 336 (e.g., registers; filters; output controllers; memory controllers) configured to generate the control signals 332 in response to the power and/or data signals 324. The at least one microcontroller 334 can comprise at least one application-specific integrated circuit (ASIC) microcontroller, digital signal processing (DSP) microcontroller, and/or microcontroller core.

In certain implementations, the stimulation circuitry 340 is configured to respond to the control signals 332 by providing stimulation signals to the recipient's body 305. For example, for a cochlear implant auditory prosthesis, the stimulation circuitry 340 can comprise a stimulator unit 120 and an elongate electrode assembly 118 comprising a plurality of longitudinally spaced intra-cochlear electrical stimulating contacts (e.g., electrodes) that configured to deliver electrical stimulation (e.g., current) to the recipient's cochlea. In certain other implementations, the stimulation circuitry 340 is configured to respond to the control signals 332 by providing at least one medicament to the recipient's body 305. For example, for an implantable micropump system, the stimulation circuitry 340 can comprise at least one flow control element (e.g., peristaltic pump; valve) in fluid communication with at least one reservoir containing at least one medicament, the at least one flow control element configured to selectively allow or inhibit (e.g., pump selectively turns on and off; valve selectively opens and closes) the at least one medicament to flow (e.g., through at least one cannula) from the at least one reservoir to the recipient's body 305 in response to the control signals 332. In certain implementations, as schematically illustrated by FIG. 3, the control circuitry 330 and the stimulation circuitry 340 are both components of functional circuitry of the apparatus 300.

In certain implementations, the external device 250 is configured to transmit a reset signal 352 to the apparatus 300 by imparting a predetermined temporal profile 400 (e.g., time-varying modulations) on the electromagnetic carrier waves transmitted by the external communication circuitry 260 via the transcutaneous wireless communication link 322. For example, as schematically illustrated by FIG. 3, the external functional circuitry 270 can comprise a reset sequence generator 274 configured to impart (e.g., apply) a predetermined temporal profile 400 on the transmitted electromagnetic carrier waves to communicate a reset signal 352 to the apparatus 300. The reset circuitry 350 is configured to monitor the electromagnetic carrier waves received by the communication circuitry 320 for the predetermined temporal profile 400 indicative of a reset signal 352.

There are numerous parameters of the communication circuitry 320 that can affect correct reception of the electromagnetic carrier waves via the transcutaneous wireless communication link 322. In certain implementations in which these parameters can be altered by firmware of the apparatus 300, the predetermined temporal profile 400 can be defined to be sufficiently simple that changes to any of the parameters of the communication circuitry 320 do not affect correct interpretation (e.g., decoding) by the reset circuitry 350 of whether the received electromagnetic carrier waves exhibits the predetermined temporal profile 400 indicative of a reset signal 352.

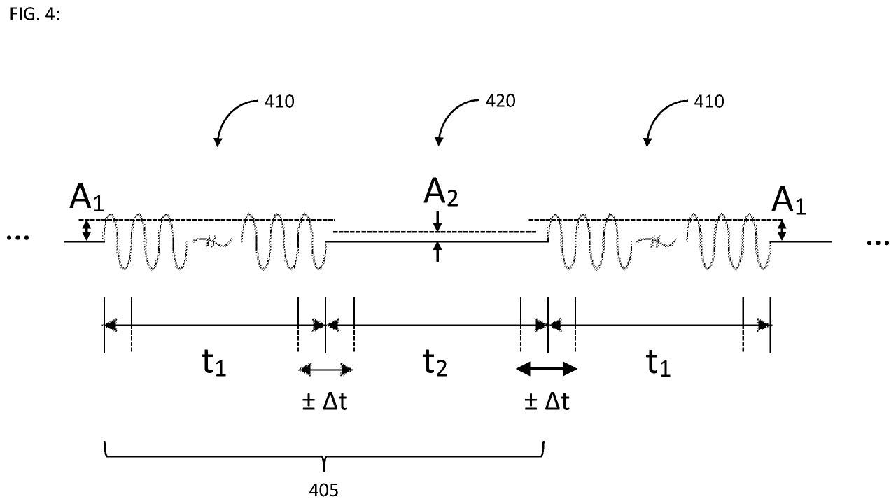

FIG. 4 schematically illustrates an example predetermined temporal profile 400 (e.g., a time-varying modulation pattern) indicative of a reset signal 352 in accordance with certain implementations described herein. The example predetermined temporal profile 400 of FIG. 4 comprises at least a predetermined number of consecutive cycles 405 (e.g., at least 128 consecutive on/off keying cycles), each cycle 405 comprising a first portion 410 having a first magnitude greater than a first predetermined threshold value A₁ over a first predetermined temporal span t₁ (e.g., an “on” cycle portion), and a second portion 420 immediately following the first portion 410, the second portion 420 having a second magnitude less than a second predetermined threshold value A₂ over a second predetermined temporal span t₂ (e.g., an “off” cycle portion). For example, the first predetermined threshold value A₁ can be greater than or equal to the second predetermined threshold value A₂ and/or the first predetermined temporal span t₁ can be substantially equal to the second predetermined temporal span t₂ (e.g., 1 millisecond). As schematically illustrated by FIG. 4, the first portion 410 comprises a substantially continuous waveform (e.g., pulse) having a carrier frequency (e.g., 5 MHz; 22.7 MHz; 2.4 GHz) and the second portion 420 comprises an absence of a waveform. The predetermined temporal profile 400 can comprise a minimum of 128 consecutive cycles, each cycle comprising a 1-ms RF signal transmission and a 1-ms pause or absence of the RF signal. Other predetermined temporal profiles 400 are also compatible with various implementations described herein (e.g., any sequence of on/off keying at any frequency), with the reset circuitry 350 configured to decode (e.g., recognize) the predetermined temporal profile 400 imparted by the external device 250 on the electromagnetic carrier waves without the decoding being influenced by the various parameters of the communication circuitry 320.

In certain implementations, the reset circuitry 350 is configured to receive information from the communication circuitry 320 regarding the temporal profile (e.g., time-varying modulations) of the electromagnetic carrier waves received by the communication circuitry 320 from the external device 250 via the transcutaneous wireless communication link 322. The reset circuitry 350 is further configured to evaluate whether the received temporal profile satisfies the predetermined criteria indicative of a reset signal 352, and if the criteria are satisfied by the received temporal profile, to reset the control circuitry 330 and/or the stimulation circuitry 340 to the default operational state. For example, the reset circuitry 350 is configured to recognize whether the received temporal profile has at least the predetermined number of cycles 405, each cycle having the first portion 410 and the second portion 420 with magnitudes and temporal spans within predetermined tolerances (e.g., temporal spans within a time ±Δt of the corresponding predetermined temporal spans) of the predetermined temporal profile 400.

In certain implementations, the reset circuitry 350 comprises hardware that exclusively decodes the received temporal profile such that other components of the apparatus 300 do not affect (e.g., influence) the decoding. For example, the control circuitry 330 and the reset circuitry 350 can be portions of different microcontrollers (e.g., ASIC microcontrollers). For another example, the control circuitry 330 can comprise a first portion of an ASIC microcontroller and the reset circuitry 350 can comprise a second portion of the ASIC microcontroller, the second portion dedicated to responding to the reset signals 352 by resetting the control circuitry 330 and/or the stimulation circuitry 340 to the default operational state. In certain implementations in which the external device 250 transmits the reset signal 352 simultaneously over multiple communication channels of the transcutaneous wireless communication link 322, the reset circuitry 350 can comprise a low-level hardware detection circuit of the apparatus 300.

In certain implementations, the reset circuitry 350 is configured to, upon decoding the received temporal profile as satisfying the criteria of the predetermined threshold profile 400 indicative of the reset signal 352, transmit a reset command 354 to the control circuitry 330 and/or the stimulation circuitry 340. In response to the reset command 354, the control circuitry 330 and/or the stimulation circuitry 340 enter a corresponding default operational state (e.g., a reset state; a safe state). For example, the default operational state of the control circuitry 330 can have the at least one microcontroller 334 and all of the other digital control circuitry 336 (e.g., registers; filters; output controllers; memory controllers) in their states corresponding to when the control circuitry 330 is without power but is configured for normal operation upon power being provided.

In certain implementations, as schematically illustrated by FIG. 3, the apparatus 300 further comprises at least one power source 360 within the at least one housing 310, the power source 360 configured to store power received by the communication circuitry 320 and to provide at least some of the power at least to the control circuitry 330 and/or the stimulation circuitry 340. For example, the at least one power source 360 can comprise at least one power storage device 362 (e.g., at least one battery; at least one capacitor) and at least one switch 364 (e.g., analog switch; digital switch) configured to controllably engage the at least one power storage device 362 with the control circuitry 330 and/or the stimulation circuitry 340 and to controllably disengage the at least one power storage device 362 from the control circuitry 330 and/or the stimulation circuitry 340.

In certain implementations, the control circuitry 330 is responsive to the reset command 354 by disengaging the at least one power storage device 362 from the control circuitry 330 and/or the stimulation circuitry 340. For example, as schematically illustrated by FIG. 3, the control circuitry 330 can comprise power control circuitry 338 configured to transmit power control signals 339 to the at least one power source 360, and the at least one switch 364 can respond to the power control signals 339 by engaging and/or disengaging the at least one power storage device 362 from the control circuitry 330 and/or the stimulation circuitry 340. In response to the reset command 354, the power control circuitry 338 can transmit power control signals 339 that are configured such that the at least one switch 364 responds by disengaging the at least one power storage device 362 from the control circuitry 330 and/or the stimulation circuitry 340. In certain other implementations, the at least one power source 360 is responsive to the reset command 354 by disengaging the at least one power storage device 362 from the control circuitry 330 and/or the stimulation circuitry 340. For example, the at least one switch 364 can be configured to receive the reset command 354 from the reset circuitry 350 and to respond to the reset command 354 by disengaging the at least one power storage device 362 from the control circuitry 330 and/or the stimulation circuitry 340.

In certain implementations, the reset circuitry 350 is configured to perform the resetting of the control circuitry 330 and/or the stimulation circuitry 340 to the default operational state regardless of the current operational state of the control circuitry 330 and/or the stimulation circuitry 340. For example, upon receipt of the reset signal 352 from the external device 350, the reset circuitry 350 can transmit the reset command 354 to the control circuitry 330, the stimulation circuitry 340, and/or the at least one power source 360, which are configured to respond to the reset command 354 (e.g., by entering the default operational state) regardless of the current operational state of the control circuitry 330 and/or the stimulation circuitry 340 (e.g., regardless of whether the control circuitry 330 and/or the stimulation circuitry 340 are malfunctioning). The control circuitry 330 and/or the stimulation circuitry 340 of certain implementations are configured to be unable to render the reset circuitry 350 inoperable.

The reset circuitry 350 of certain implementations provides a firmware independent reset of the apparatus 300 that can be used as a “fail/safe” reset mechanism. For example, while implantable medical devices (e.g., cochlear implants) undergo a rigorous risk assessment process that endeavors to identify and mitigate any potential failures of the medical device that could expose the recipient to harm and/or inconvenience, such risk assessment processes cannot completely guarantee to include all potential failures. In addition, while risk assessment processes are typically based on single fault failures based on an assumption that two independent failures rarely occur simultaneously, multiple fault failures have a non-zero probability of occurring. The reset circuitry 350 of certain implementations is configured to provide a firmware independent reset of the apparatus 300 that can operate even if an unforeseen event or a multiple fault failure occurs, since the reset does not rely on the microprocessor executed firmware. By having the reset signal communicated via the same communication link as are the power and/or data signals (e.g., “piggybacking”), certain implementations described herein do not add significant overhead on operations and/or provide reliable triggering of the reset operation (e.g., avoids erroneous triggering). In certain such implementations, such “piggybacking” provides an extra layer of safety by ensuring that the reset communication mechanism works when needed. If the communications mechanism between the external device 250 and the apparatus 300 fails for some reason (e.g., failure of the communication circuitry 260 and/or the communication circuitry 320), the failure will become apparent due to a failure of normal operation of the apparatus 300, even before a reset of the apparatus 300 is to be triggered. For example, the apparatus 300 receiving and responding to stimulation commands from the external device 250 would cease—which is not necessarily unsafe, but would be noticed by the recipient. In contrast, a reset communication mechanism that is separate from the power and/or data communication link would lie dormant until such time as a reset is to be triggered, and a failure of the reset communication mechanism at some time prior to its intended use would go unnoticed. By having the reset operation disconnect the power source from the other circuitry of the apparatus 300, certain implementations described herein provide a heightened safety level while allowing any failures that invoke the reset operation to be noticed by a user (e.g., recipient; healthcare provider; diagnostic technician).

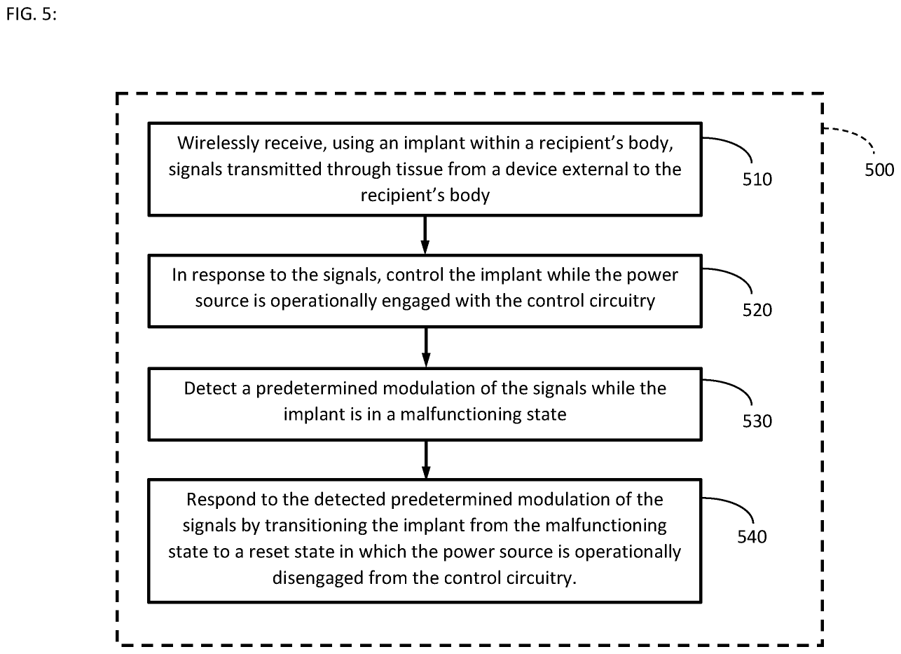

FIG. 5 is a flow diagram of an example method 500 in accordance with certain implementations described herein. While the method 500 is described by referring to some of the structures of the example apparatus 300 of FIG. 3, other apparatus and systems with other configurations of components can also be used to perform the method 500 in accordance with certain implementations described herein.

In an operational block 510, the method 500 comprises wirelessly receiving, using an implant within a recipient's body (e.g., apparatus 300), signals transmitted through tissue from a device external to a recipient's body (e.g., external device 250), the implant comprising control circuitry 330 and an internal power source 360.

In an operational block 520, the method 500 further comprises, in response to the signals, controlling the implant while the implant is in at least one functional state in which the internal power source 360 is operationally engaged with the control circuitry 330. For example, the implant in the at least one functional state can be operating normally. In an operational block 530, the method 500 further comprises detecting, using the implant, a predetermined modulation of the signals while the implant is in a malfunctioning state. For example, the implant in the malfunctioning state can be operating abnormally. Detecting the predetermined modulation of the signals can comprise decoding modulations of the signals using firmware of the implant that is dedicated to said decoding.

In an operational block 540, the method 500 further comprises responding to the detected predetermined modulation of the signals by transitioning the implant from the malfunctioning state to a reset state in which the internal power source is operationally disengaged from the control circuitry.

In certain implementations, the method 500 further comprises subsequently operationally re-engaging the power source 360 with the control circuitry 330 in response to the signals.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of various devices, various implementations described herein can be incorporated in a variety of other suitable devices, methods, and contexts. More generally, as can be appreciated, certain implementations described herein can be used in a variety of implantable medical device contexts that can benefit from certain attributes described herein.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein but should be defined only in accordance with the claims and their equivalents.

1. An apparatus comprising:
at least one housing configured to be implanted within a recipient's body;
communication circuitry within the at least one housing, the communication circuitry configured to wirelessly communicate, via a transcutaneous wireless communication link, with a device external to the recipient's body;
control circuitry within the at least one housing, the control circuitry configured to generate control signals in response to power and/or data signals received via the transcutaneous wireless communication link;
stimulation circuitry within the at least one housing, the stimulation circuitry configured to respond to the control signals by providing stimulation and/or at least one medicament to the recipient's body; and
reset circuitry within the at least one housing, the reset circuitry configured to respond to reset signals received via the transcutaneous wireless communication link by resetting the control circuitry and/or the stimulation circuitry to a default operational state.

2. The apparatus of claim 1, further comprising at least one power source within the at least one housing, the at least one power source comprising at least one power storage device configured to store power received by the communication circuitry and to provide at least some of the power at least to the control circuitry and/or the stimulation circuitry.

3. The apparatus of claim 2, wherein the at least one power source is responsive to the reset signals by disengaging the at least one power storage device from the control circuitry and/or the stimulation circuitry.

4. The apparatus of claim 2, wherein the control circuitry is responsive to the reset signals by disengaging the at least one power storage device from the control circuitry and/or the stimulation circuitry.

5. The apparatus of claim 2, wherein the power storage device comprises at least one battery.

6. The apparatus of claim 1, wherein the reset circuitry is configured to perform said resetting the control circuitry and/or the stimulation circuitry to the default operational state regardless of a current operational state of the control circuitry and/or the stimulation circuitry.

7. The apparatus of claim 1, wherein the control circuitry and/or the stimulation circuitry are configured to enter the default operational state upon being reset by the reset circuitry regardless of a current operational state of the control circuitry and/or the stimulation circuitry.

8. The apparatus of claim 1, wherein the control circuitry comprises a first portion of an application-specific integrated circuit (ASIC) microcontroller and the reset circuitry comprises a second portion of the ASIC microcontroller, the second portion dedicated to responding to the reset signals by resetting the control circuitry and/or the stimulation circuitry to a default operational state.

9. The apparatus of claim 1, wherein the reset signals have a predetermined temporal profile comprising a predetermined number of consecutive cycles, each cycle comprising:
a first portion having a first magnitude greater than a first predetermined threshold value over a first predetermined temporal span; and
a second portion immediately following the first portion, the second portion having a second magnitude less than a second predetermined threshold value over a second predetermined temporal span.

10. The apparatus of claim 7, wherein the first predetermined threshold value is greater than or equal to the second predetermined threshold value.

11. The apparatus of claim 7, wherein the first predetermined temporal span is substantially equal to the second predetermined temporal span.

12. The apparatus of claim 1, wherein the communication circuitry comprises at least one internal radio-frequency (RF) antenna in operative communication with at least one external RF antenna of the device to form the transcutaneous wireless communication link.

13. The apparatus of claim 1, wherein the communication circuitry comprises at least one internal magnetic induction (MI) antenna in operative communication with at least one external MI antenna of the device to form the transcutaneous wireless communication link.

14. The apparatus of claim 1, wherein the control circuitry is configured to be unable to render the reset circuitry inoperable.

15. The apparatus of claim 1, wherein the apparatus comprises an acoustic prosthesis.

16. A method comprising:
wirelessly receiving, using an implant within a recipient's body, signals transmitted through tissue from a device external to a recipient's body, the implant comprising control circuitry and an internal power source;
in response to the signals, controlling the implant while the implant is in at least one functional state in which the internal power source is operationally engaged with the control circuitry;
detecting, using the implant, a predetermined modulation of the signals while the implant is in a malfunctioning state; and
responding to the detected predetermined modulation of the signals by transitioning the implant from the malfunctioning state to a reset state in which the internal power source is operationally disengaged from the control circuitry.

17. The method of claim 14, wherein the implant in the at least one functional state operates normally and the implant in the malfunctioning state operates abnormally.

18. The method of claim 14, further comprising subsequently operationally re-engaging the internal power source with the control circuitry in response to the signals.

19. The method of claim 14, wherein said detecting comprises decoding, using firmware of the implant, modulations of the signals, the firmware dedicated to said decoding.

20. A system comprising:
a first portion configured to be worn externally on a recipient's body, the device configured to generate electromagnetic carrier waves with time-varying modulations; and
a second portion configured to be implanted within the recipient's body, the second portion configured to transcutaneously receive at least a portion of the electromagnetic carrier waves from the first portion, the second portion comprising:
at least one microprocessor configured to execute firmware configured to control operation of the second portion in response to the electromagnetic carrier waves received from the first portion; and
reset circuitry configured to operate independently from operation of the firmware, the reset circuitry further configured to monitor the time-varying modulations for a pattern indicative of a reset signal from the first portion and to respond to detection of the pattern on the electromagnetic carrier waves by resetting the at least one microprocessor.

21. The system of claim 20, wherein the firmware is unable to prevent and/or interfere with the resetting of the at least one microprocessor.

22. The system of claim 20 , wherein the pattern comprises an on/off keying pattern.

23. The system of claim 20, wherein the first portion comprises an external component of an auditory prosthesis system and the second portion comprises an implanted component of the auditory prosthesis system.
